SEQUENCE LISTING (9 sequences)

SEQ ID NO: 1 - RNA, 19 nt, Artificial Sequence
  Description of Artificial Sequence Synthetic oligonucleotide
gggaaaagaaagaauguau

SEQ ID NO: 2 - RNA, 19 nt, Artificial Sequence
  Description of Artificial Sequence Synthetic oligonucleotide
gggagaaugucuuucaaga

SEQ ID NO: 3 - RNA, 19 nt, Artificial Sequence
  Description of Artificial Sequence Synthetic oligonucleotide
ggcccaggugguaaucauu

SEQ ID NO: 4 - RNA, 19 nt, Artificial Sequence
  Description of Artificial Sequence Synthetic oligonucleotide
gguuuugaacgaagaaugu

SEQ ID NO: 5 - RNA, 19 nt, Artificial Sequence
  Description of Artificial Sequence Synthetic oligonucleotide
gguugauacaggcaauacu

SEQ ID NO: 6 - RNA, 19 nt, Artificial Sequence
  Description of Artificial Sequence Synthetic oligonucleotide
ggagagguuuguucccuga

SEQ ID NO: 7 - RNA, 19 nt, Artificial Sequence
  Description of Artificial Sequence Synthetic oligonucleotide
ggaggacugggaauacaua

SEQ ID NO: 8 - RNA, 19 nt, Artificial Sequence
  Description of Artificial Sequence Synthetic oligonucleotide
ggaaugugaaaaagcuaca

SEQ ID NO: 9 - RNA, 19 nt, Artificial Sequence
  Description of Artificial Sequence Synthetic oligonucleotide
ggaaaacaucaagaacaau

(19) United States
(12) Patent Application Publication
(10) Pub. No.: US 2006/0217308 A1
(43) Pub. Date: Sep. 28, 2006

(54) Compositions for the treatment of tumor pathologies, comprising internalin B (ln1B) of Listeria monocytogene protein or a fragment thereof

(76) Inventors: Pascale Cossart, Paris (FR); Esteban Veiga, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/372,462

(22) Filed: Mar. 10, 2006

Related U.S. Application Data
(60) Provisional application No. 60/659,887, filed on Mar. 10, 2005.

(51) Int. Cl. A61K 38/17

(52) U.S. Cl. 514/12

(57) ABSTRACT

The pathway by which Met is degraded after interacting with In1B, in response to Listeria monocytogenes infection, provides In1B or In1B fragments or peptides, preferably those that interfere with the interaction between In1B and Met, as new cancer treatments that can achieve rapid down-regulation and subsequent degradation of Met. In addition, this pathway highlights methods of screening for other compounds that modulate the interaction between Met and In1B and may also be used therapeutically.

### CROSS-REFERENCE TO RELATED APPLICATIONS

The application claims the benefit of priority of U.S. Provisional Application No. 60/659,887 (attorney docket number 03495-6107), filed Mar. 10, 2005, which is incorporated herein by reference.

### FIELD OF THE INVENTION

This invention relates to the field of treatments for cancer.

### BACKGROUND OF THE INVENTION

The receptor tyrosine kinase (RTK), Met, also known as hepatocyte growth factor receptor is involved in diverse cellular functions such as scattering, invasion, proliferation, morphogenesis and angiogenesis (1). It is also implicated in a large number of human tumours, correlating closely with metastasis and poor prognosis (2, 3). These tumorigenic activities occur when Met is overexpressed, or when Met signalling after ligand (HGF) contact is not downregulated (4). Several polypeptide fragments and variants have been found to be activators of Met. See US 2004/0236073, published Nov. 25, 2004; European Patent EP 0 981 620, published Feb. 25, 2004, which are herein incorporated by reference. In contrast, cells can stop signalling downstream of Met, as well as other RTKs, through ligand-dependent endocytosis and subsequent degradation of activated receptor (4-6).

Ubiquitination is a post-translational modification of proteins in which the modifier is itself a polypeptide that is conjugated to the target proteins by forming an isopeptide bond between the carboxyl terminus of ubiquitin and a lysine side chain(s) in the target protein (7, 8). Protein modification by ubiquitin occurs in three successive steps mediated by the enzymes E1 (activating enzyme), E2 (conjugating enzyme), and E3 (ubiquitin ligase). Ubiquitin molecules can form poly-ubiquitin chains conjugated to target proteins that are normally recognized and degraded by the proteasome (7, 9). However, covalent attachment of only one ubiquitin module (mono-ubiquitin) or multiple mono-ubiquitin modules does not result in proteasomal degradation of modified protein, but has been shown to modulate biological functions such as endocytosis and transport to lysosomes (7, 10, 11). Recent studies have shown that ligand-dependent endocytosis of RTKs and other membrane proteins is triggered by mono-ubiquitination of the receptor, mediated by the ubiquitin ligase Cbl (6, 12-18). This picture is more complicated in the case of Met, whose mechanism of degradation after endocytosis is unclear because both proteasomal (19) or lysosomal (20) pathways have been described as degradation mechanisms. Furthermore, Met-ubiquitination has been reported to be monoubiquitination (21) or polyubiquitination (15, 20), even though Cbl is the ubiquitin ligase that recognizes Met (13, 15, 22).

L. monocytogenes is an intracellular pathogen able to use Met as receptor to promote its internalization into normally non-phagocytic epithelial cells (23-25). The bacterial protein that interacts with Met is a protein of the internalin family called In1B (25-28). The interaction between In1B and Met triggers the tyrosine phosphorylation of Met and the recruitment to activated Met of different proteins e.g. Gab1, Shc, and Cbl, among others (29-31). Soluble In1B behaves as a growth factor i.e. triggering similar signalling events and membrane ruffles (30). Remarkably, Cbl is recruited during the first minute following In1B addition to cultured cells and Met becomes rapidly phosphorylated (31). Moreover, latex beads coated with In1B are also able to enter into cells, indicating that In1B is sufficient to promote phagocytosis (24, 32).

Agents that inhibit Met signalling represent an important therapeutic avenue for the treatment of a variety of malignant tumors. For example, antibiotics of the Geldamycin family that down-regulate the expression of Met are in clinical trials for cancer treatments (55, 56). In addition, the anticancer activities of antagonistic antibodies against Met and neutralizing antibodies against Met ligand (HGF) are also important potential anticancer therapies. There is a need in the art for new treatments for cancer based on the activities of Met.

### SUMMARY OF THE INVENTION

The invention fulfills this need by illuminating a pathway by which Met is degraded after interacting with In1B, in response to Listeria monocytogenes infection. Listeria uses the surface protein In1B to invade a variety of cell types, while In1B interacts with the hepatocyte growth factor receptor, Met, on mammalian cells. This interaction is critical for infection. The examples provided herein demonstrate the basis for the invention by showing that rapidly after contact, In1B induces the mono-ubiquitination and endocytosis of Met and its subsequent degradation in a proteasome-independent manner. Moreover, overexpression or down-regulation of Cbl, the ubiquitin ligase that ubiquitinates Met, respectively increases or decreases bacterial invasion in epithelial cells, and RNAi-mediated knock down of major components of the endocytic machinery inhibits bacterial entry. Thus, the invention relies on the discovery that a bacterium induces and requires the ubiquitin-dependent endocytosis of its receptor to invade host mammalian cells. Furthermore, the invention is based on the knowledge that the In1B-induced degradation of Met is an extremely rapid process, being observed one minute after the addition of In1B, in contrast to activation with HGF, which causes a similar rate of Met degradation after two to five hours.

Based on this understanding, the invention provides In1B or In1B fragments or peptides, preferably those that interfere with the interaction between In1B and Met, as new cancer treatments that can achieve rapid down-regulation and subsequent degradation of Met. In addition, the invention provides screening tests to determine which compounds and In1B variants modulate the interaction between Met and In1B and/or interfere with Met signalling.

### BRIEF DESCRIPTION OF THE DRAWINGS

FIG. 1 shows that Listerial protein In1B induces ubiquitination of Met. (A) Listeria monocytogenes EGD (BUG600) (MOI 25) or purified In1B (5 nM) were added (+) for two minutes to HeLa cells. Samples were immunoprecipitated against Met and blotted against ubiquitin using P4D1 monoclonal antibody. (B) In1B was added to HeLa cells for two minutes and samples were re-immunoprecipitated against Met. Samples were blotted against ubiquitin (Ub) or Met. (C) Increasing amounts of In1B were added to HeLa cells (1 to 6 corresponding to 0, 0.22, 0.44, 0.88, 1.75, 3, 5 nM respectively) for two minutes. Extracts were immunoprecipitated against Met and blotted against Met or ubiquitin (Ub). (D) As C, but 1 to 6 correspond to 0, 3.5, 10, 20, 40, 80 nM In1B, respectively. (E) 5 nm of purified In1B was added to HeLa cells for the indicated times. Cell extracts were immunoprecipitated (IP) against Met and blotted (WB) against Met or ubiquitin (Ub). At the left of all panels the molecular weight markers are indicated in kDa. In C and D, 8% acrylamide gels were used, while 10% acrylamide gels were used in A, B, and E.

FIG. 2 depicts the degradation and de-novo synthesis of Met. (A) HeLa cells were treated at the indicated times with 5 nM In1B and cellular extracts were blotted against Met. (B) Cells were treated as in A, but blotted at the same time with anti-Met antibody and Streptavidin. Stars indicate nonspecific bands recognized by streptavidin and the square indicates Met. (C) The experiment was performed as in A, but cells were pre-treated with 100 mg/ml of cycloheximide for 90 minutes before addition of In1B. (D) This experiment was performed as in A, but In1B was removed three minutes after it was added. Actin is shown as the loading control in A, C and D. The molecular mass of the markers is indicated in kDa.

FIG. 3 demonstrates that In1B induces mono-ubiquitination of Met. (A) In1B was added (+) to HeLa cells. Lysates were immunoprecipitated using C12 rabbit polyclonal anti-Met antibody and blotted against Met (DL21 mAb). The same membrane was stripped and blotted against ubiquitin using FK1, a mAb that recognizes poly-ubiquitinated proteins, or P4D1, a mAb that recognizes both poly- and mono-ubiquitinated proteins. (B) Total cell extracts from Hela cells treated with 1 mM epoximycin (lane 2) or 10 mM clasto-lactacystin-β-lactone (lane 3) for 60 minutes, or non-treated cells (lane 1) were blotted against poly-ubiquitin (FK1) or actin. (C) ubiquitin ladder was blotted with FK1 or P4D1. The number of ubiquitin modules is indicated on the right. The molecular weight of protein markers is shown in the left of the panels, in kDa. In D and E, Hela Cells were transfected with siRNA to knock down Cbl expression (RNAi), or with RNA control (Control). (D) Cellular Cbl and actin were detected by immunoblotting. (E) Cell lysates were immunoprecipitated (IP) with Met antibody and Met precipitates were immunodetected (WB) with anti ubiquitin (Ub) or anti Met antibodies.

FIG. 4 demonstrates the nature of Met degradation. (A) Hela cells were treated (+) with 1 mM epoximycin, 10 mM clasto-lactacystin-β-lactone (lactone), 20 mM ammonium chloride, or 5 μM bafilomycin A1 (Baf-A1) for 90 minutes before addition of In1B. Cell extracts were loaded in a SDS-PAGE gel followed by immunoblot with anti-Met antibody. Actin is showed as a loading control. (B) Surface exposed proteins of Hela cells were biotinylated (Biot +) before addition of In1B. In1B was added for two minutes to allow internalization of Met, then cells were washed and biotin was removed (see methods and text) from the surface. Samples were precipitated with neutravidin agarose and blotted against Met.

FIG. 5 shows degradation of Met during Listeria infection. (A) HeLa cells were infected during the indicated times with Listeria (BUG600) (50 MOI), and total cellular extracts were blotted against Met. (B) HeLa cells were treated or not with cyclohexamide (cycloh). Then, purified 5 nM In1B or Listeria (BUG600) (50 MOI) was added to the cell cultures for 15 minutes. Total cell extracts were immunodetected with anti-Met or anti-actin antibodies. (C) Hela cells were infected for ten minutes with Listeria strain BUG 1641. Bacteria were detected with anti-Listeria rabbit serum (panel 1) and cellular Met was detected with DL-21 mAb (panel 2). The merge image is shown in panel 3 showing bacteria green, Met red, and the DAPI stained nucleus in blue. Magnification squares are shown for a better observation of Met-Listeria colocalization.

FIG. 6 demonstrates the role of Cbl in Listeria infection. (A) Hela cells were infected for three minutes with Listeria strain BUG 1641. Bacteria were detected with anti Listeria rabbit serum (green, panel 1) and Ha-tagged Cbl was detected with anti-Ha antibody (red, panel 2). The merge image is shown in panel 3. Magnification squares are shown for a better view of Cbl-Listeria colocalization. In B and C, Hela Cells were transfected with short interfering RNA to knock down Cbl expression (RNAi), or with RNA control (Control). (B) Cellular Cbl and actin were detected by immunoblotting. (C) Transfected cells were infected with Listeria monocytogenes BUG600 in a typical gentamicin survival assay. The graph shows the level of infection on RNAi cells compared to control cells. (D) Hela cells expressing Ha-Cbl, Ha-v-Cbl, or the parental plasmid pMT2SM-Ha (Control) were infected with Listeria and differential immunofluorescent labelling were performed. The number of intracellular versus extracellular bacteria was counted after infection and the percentage of intracellular bacteria was represented as a comparison to that observed in the control. (E) HeLa cells were transfected with Ha-tagged ubiquitin (Ub), ubiquitin K29,48,63R (mono-ub), or the parental plasmid. Transfected cells were infected with Listeria monocytogenes BUG600 and typical differential immunofluorescent labelling was performed. The percentage of intracellular bacteria was represented compared to that observed in cells transfected with the control plasmid. For each differential immunofluorescent labelling assay, four different independent experiments were performed and in every experiment a minimum of 100 individual cells were examined.

FIG. 7 depicts the role of endocytosis machinery in Listeria entry. (A) Hela Cells were transfected with siRNA to knock down the expression of indicated proteins or with control RNA, left and right lanes respectively in each panel. The expression of the indicated proteins (upper pannels) and actin (down pannels) was followed by immunoblotting. (B) Transfected cells were infected with Listeria monocytogenes BUG600 in a typical gentamicin survival assay. The graph shows the level of infection on RNAi cells compared to control cells.

FIG. 8 depicts the kinetics of phosphorylation and ubiquitination of Met. 5 nm of purified In1B was added to HeLa cells for the indicated times. Cell extracts were immunoprecipitated (IP) against Met and blotted (WB) against phospho-tyrosine (P-Tyr), Met or ubiquitin (Ub).

FIG. 9 depicts immunodetection of Met from total cell lysates. Serum-containing media (1%) with HGF (1 nM), HGF (1 nM)+InIB (5 nM), or without either HGF or InIB (control) was added to HeLa cells. Cells were recovered at the indicated times after addition of HGF, and HGF+InIB, resuspended in SDS-PAGE sample buffer, and immediately boiled for 5 minutes. The results show immunodetection of Met from total cell lysates. Actin is showed as a loading control.

FIG. 10 provides a model of In1B-mediated Listeria internalization. Soluble and bacterial surface attached In1B interact with Met, then Met autophosphorylates and recruits Cbl, which mono-ubiquitinates Met and induces its endocytosis. Finally, the endocytic machinery allowing Met-endocytosis is used by Listeria to penetrate into epithelial cells.

### DETAILED DESCRIPTION OF THE INVENTION

The invention is based on the discovery that a bacterium can trigger and exploit the monoubiquitin-dependent endocytosis machinery, to invade non-phagocytic mammalian cells. First, following In1B-dependent activation, Met becomes mono-ubiquitinated, and as with other RTKs, mono-ubiquitination of Met triggers its endocytosis. In addition, Listeria invasion depends on the endocytic machinery and Cbl, the ubiquitin ligase that ubiquitinates Met and other RTKs (5, 13-15), is needed for this invasion process. Overexpression of Cbl enhances, up to eight times, bacterial entry and down-regulation of Cbl, while dominant negative proteins or siRNA strongly inhibit bacterial entry, just as over expression or down-regulation of Cbl respectively increases or decreases endocytosis of RTKs (13, 14). The role of the ubiquitin is also highlighted by the ability of ubiquitin, or a ubiquitin that can only be mono-ubiquitinated (17), to increase bacterial entry by three-fold.

RNAi knock-down expression of different proteins involved in ligand induced RTK endocytosis including clathrin, eps15, Gbr2, dynamin, CiN85, CD2AP, cortactin, also inhibits Listerial entry, emphasizing the requirement of the endocytic machinery in the internalization/endocytosis of Listeria. The Examples provided herein show that the clathrin adaptor complex AP-2 is not involved in endocytosis of Listeria. Indeed, previous studies have shown that EGFR endocytosis is also independent of AP-2 (44, 45). In contrast, another clathrin adaptor complex, AP-1 (43) seems to be implicated in the entry of Listeria. Furthermore, knocking-down expression by RNAi of the clathrin adaptors GGA3 and Hrs, which are also ubiquitin-binding proteins (43), strongly inhibits Listeria endocytosis. GGA3 and AP-1 are involved in the trafficking between Golgi apparatus and endosomes (43, 46) and Hrs appears to associate with late endosomes or multivesicular bodies (47, 48), suggesting that the Golgi apparatus, endosomes, or both could provide membrane to allow the endocytosis of Listeria.

In1B-induced degradation of Met is extremely rapid, observable as soon as one minute after addition of In1B. In contrast, two or more hours are needed to achieve a similar rate of Met degradation after activation with HGF (19-21), indicating that In1B- and HGF-induced degradation of Met are different phenomena. Interestingly, Met is specifically and rapidly resynthesized after degradation, indicating that there must exist a yet unknown mechanism that couples the endocytic machinery to specific activation of the gene encoding the degraded protein.

Endocytosis is a cellular mechanism used to internalize receptors and other cargo molecules from the plasma membrane by invagination and pinching-off of membrane bound vesicles (49). In addition to macromolecules, it is also well known that large particles, such as viruses are able to get into the cell by endocytosis (50, 51). Moreover, the time required to complete assembly of the endocytic complex is proportional to the size of cargo molecules (36). The endocytosis of large particles, such as viruses, takes more time than for smaller molecules, such as transferrin. Accordingly, Cbl co-localizes with bacteria, as it is observed around activated receptor when endocytosis is slowed down (52).

The fact that bacteria, which are ten to fifty times larger than viruses, harness the endocytic machinery to invade non-phagocytic mammalian cells indicates that the size of cargo particles able to enter cells by endocytosis can be much larger than previously thought. Such large particles will also require a force to get in. An attractive candidate to generate this force is actin polymerization. Actin polymerization is indeed critical for Listeria invasion (53). It has been well established that actin polymerization during In1B-mediated entry involves the Arp2/3 complex in conjunction with proteins of WASP family shortly after In1B-mediated activation of Met (53). Interestingly, actin and actin-binding proteins play a critical role in yeast endocytosis and recent studies have pointed to a major role of the actin cytoskeleton in endocytosis in mammalian cells (38, 41). CD2AP binds Cbl and cortactin, which are able to directly activate the arp2/3 complex (42). It is now well established that dynamin can bind both directly and indirectly to activators of the Arp2/3 complex that are important for clathrin mediated endocytosis, including cortactin and the complex between N-WASP and syndapin. In addition, eps15 can recruit intersectin 1, which is also able to activate actin polymerization via activation of the arp2/3 complex (41). The clathrin coat can have a pivotal role supporting the invagination, transducing the compressive force generated by actin polymerization into pinching movement leading to fission at the neck of the phagocytic cup.

Thus, exploitation of the endocytic machinery can be a general mechanism used by bacteria to infect mammalian cells, as well as contributing to the formation of tumors.

In an embodiment of the invention, a method for treating a carcinogenic tumor in a mammal is provided. The method comprises administering a composition comprising In1B or a fragment of In1B to a mammal suffering from cancer, achieving degradation of Met in less than one hour, and reducing at least one carcinogenic aspect of the tumor.

In further embodiments, the carcinogenic aspects of the tumor include, but are not limited to, tumor growth, proliferation of tumor cells, angiogenesis in the tumor, invasiveness of the tumor, or dedifferentiated tumor morphology. “Tumor growth” means the expansion of the size of a solid tumor. “Proliferation of tumor cells” means the ability of the tumor cells to divide. “Angiogenesis” means the ability of a solid tumor to become vascularized. “Invasiveness” means the ability of the tumor to infiltrate non-tumor tissues. “Dedifferentiated” means that a tumor has lost the characteristics of the cell type it had before it became a tumor.

In other embodiments, the tumor can be a tumor of liver, kidney, breast, intestine, or other organs or tissues.

In yet further embodiments, the mammal that is treated by the method of the invention can be, but is not limited to, a dog, cat, cow, sheep, pig, goat, horse, monkey, or preferably, a human.

In another embodiment, the invention provides an anticancer treatment comprising a pharmaceutical composition comprising In1B or a fragment of In1B. In these embodiments of the invention, In1B or fragments of In1B are administered to the mammal. Fragments of In1B include, but are not limited to, peptides or small proteins derived from the GW domain or LRR domain of In1B. In preferred embodiments, the fragments are those described in (57), which are shown to be able to interact with Met and activate it. The LRR domain includes, but is not limited to, amino acids 76-248 of In1B. The GW domains include, but are not limited to, amino acids 392-630 of In1B. These domains, or subfragments of them, such as amino acids 464-630 of the GW domains, are also alternative embodiments for use in the claimed methods.

The In1B or a fragment of In B of the invention can be formulated as a pharmaceutical composition and administered in a variety of dosage forms adapted to the chosen route of administration, including, but not limited to, orally or parenterally, by intravenous, intramuscular, topical or subcutaneous routes.

Thus, the In1B or a fragment of In1B may be systemically administered, e.g., orally, in combination with a pharmaceutically acceptable vehicle, such as an inert diluent or an assimilable edible carrier. It may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders, such as gum tragacanth, acacia, corn starch or gelatin; excipients, such as dicalcium phosphate; a disintegrating agent, such as corn starch, potato starch, alginic acid and the like; a lubricant, such as magnesium stearate; and a sweetening agent, such as sucrose, fructose, lactose or aspartame or a flavoring agent, such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac or sugar and the like. A syrup or elixir can contain the active compound, sucrose or fructose as a sweetening agent, methyl and propylparabens as preservatives, a dye and flavouring, such as cherry or orange flavor. Of course, any material used in preparing any unit dosage form should be pharmaceutically acceptable and substantially non-toxic in the amounts employed. In addition, the active compound can be incorporated into sustained-release preparations and devices.

The active compound can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and freeze-drying techniques, which yield a powder of the active ingredient plus any additional desired ingredient present in the previously sterile-filtered solutions.

For topical administration, the In1B or a fragment of In1B can be applied in pure form, i.e., when they are liquids. However, it will generally be desirable to administer it to the skin as compositions or formulations, in combination with a dermatologically acceptable carrier, which can be a solid or a liquid.

Useful solid carriers include finely divided solids, such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants, such as fragrances and additional antimicrobial agents, can be added to optimize the properties for a given use. The resultant liquid compositions can be applied from absorbent pads, used to impregnate bandages and other dressings, or sprayed onto the affected area using pump-type or aerosol sprayers.

Thickeners, such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the In1B or a fragment of In1B can be determined by correlating their in vitro activity, and in vivo activity in animal models, such as murine or dog models. The therapeutically effective amount of compound necessarily varies with the subject and the tumor to be treated. For example, doses of 10 μg to 1 g, 25 to 500 μg, 50 to 250 μg, 75 to 150 μg, or 30 to 50 μg can be used. The desired dose can conveniently be presented in a single dose or as divided doses administered at appropriate intervals, for example, as two, three, four or more sub-doses per day.

In another embodiment of the invention, a method of increasing the degradation rate of Met is provided. In this embodiment, a cell that contains Met is exposed to a composition comprising In1B or a fragment of In1B and the degradation rate of Met is increased in the cell. By decreasing the degradation rate of Met, certain characteristics of carcinogenic cells grown in culture may be modified. For example, but not to the exclusion of other characteristics, the rate of proliferation or degree of invasiveness or dedifferentiation of such cells may be modified to achieve a particular set of circumstances under which to perform experiments.

In yet another embodiment, the invention provides a method of screening for compounds that modulate the interaction between Met and In1B, comprising (A) adding a compound to a cultured cell; (B) incubating the cell for different times; (C) analyzing the Met protein in the cell after the different times of incubation; (D) determining which compounds alter the rate of degradation of Met in the cell; and (E) selecting the compounds that alter the rate of degradation of Met as ones that modulate the interaction between In1B and Met.

By way of example, but without limiting any parameters of this embodiment, the following screening method is provided: Semi-confluent HeLa cells growing in 6-well culture dishes are washed and purified In1B (5 nm) and the test compound or a control are added directly to the medium, i.e. DMEM. After 1, 2, 5, 10, 15, 30, 45, and 60 minutes incubation at 37° C., the cells are resuspended directly in SDS-PAGE sample buffer and immediately boiled. The samples are then separated on an SDS-PAGE gel and blotted with anti-Met monoclonal antibodies, according to protocols known to those in the art. The rate of degradation of Met is compared between the samples obtained from incubation in the presence or absence of the test compound. Samples that indicate the rate of Met degradation has changed indicate that the interaction between In1B and Met was modulated.

In yet more embodiments of the invention, the compounds that are identified by the screening method are provided as therapeutic anti-cancer treatments.

In yet another embodiment, the invention relates to a screening test for In1B variants that can interfere with Met signalling, based on the ability of Met to cause the phosphorylation of various proteins after interacting with HGF. In one screening test, mammalian cells expressing Met are grown in multiwell culture dishes, for example, but not limited to ELISA culture dishes with 96 or 384 wells. Medium containing HGF and at least one In1B variant, or a fragment thereof, is added to the wells of the culture dish, while some wells receive only HGF, as controls. After contact is made between HGF and the Met in the cells, the amount of protein phosphorylation is detected and quantified. Wells in which the In1B variant reduces the amount of phosphorylation observed in the control well indicate that the In1B variant interferes with Met signalling.

In the screening tests of this embodiment, phosphorylation can be, but is not limited to, tyrosine phosphorylation. In the case of tyrosine phosphorylation, quantification can be achieved using commercially available anti-phosphotyrosine antibodies directed against phosphotyrosine residues. These antibodies can be obtained from several companies, including, but not limited to, Cell Signaling Technology, Inc. and Upstate Group, LLC.

In an alternative embodiment of the screening test, phosphorylation of specific proteins can be measured. These proteins include, but are not limited to, phospho-Met, phospho-ERK, phospho-STAT3, and phospho-46JNK. Antibodies to detect phosphorylation of these proteins are also commercially available, for example from PhosphoSolutions, Inc.

In both of these embodiments of a screening test, the ability of In1B variants to reduce either total protein phosphorylation or the phosphorylation of specific proteins is indicative of the ability of the In1B variants to interfere with Met signalling.

In addition, the screening tests provided in these embodiments can be supplemented with a confirmation test in which In1B variants are analyzed in an invasion assay. Briefly, mammalian cells are gown on a collagen matrix. HGF is added, inducing the cells to separate and invade the matrix. The ability of In1B variants to inhibit Met signalling is tested by adding In1B variants, individually, to the wells containing invasive cells. Invasion is then measured visually using a microscope, a process which can be automated. In1B variants that inhibit the ability of the cells to invade the collagen matrix are confirmed as ones that inhibit Met signalling.

Throughout this application, various publications are referenced in parentheses by number. Full citations for these references may be found at the end of the specification immediately preceding the sequence listings and the claims. The disclosure of these publications in their entireties are hereby incorporated by reference into this application to describe more fully the art to which this invention pertains.

The following examples are presented to demonstrate the methods of the present invention and to assist one of ordinary skill in using the same. The examples are not intended in any way to otherwise limit the scope of the disclosure.

### EXAMPLE 1

### Materials and Methods

Cells, bacteria and growth conditions. The human cell line used was HeLa (ATCC number CCL-2) grown as recommended by ATCC. Typically, cells were serum starved for three hours before the addition of In1B. The bacterial strains used were Listeria monocytogenes BUG600 and BUG1461. They were grown in BHI medium, which in the case BUG1641 contained 5 μg/ml of erythromycin.

Antibodies and reagents. The antibodies used were: rabbit polyclonal antibodies (pAb) anti-Met (C-12; used for immunoprecipitations), CD2AP (H-290), Grb2 (C-23), eps15 (H-896) from Santa Cruz, pAb anti Hrs (kind gift from Prof. Harald Stenmark), mAb anti-Met (DL21; used for immunoblots) from Upstate, mAb anti-ubiquitin (P4D1; Cell Signalling), mAb anti ubiquitin (FK1; Affinity), mAb anti β-actin (AC15; Sigma), mAb anti Ha (Santa Cruz), mAbs anti Cbl, AP50, Clathrin Heavy Chain, and GGA3 from BD pharmingen, pAbs anti-CIN85, dynamin II, (Calbiochem), rabbit serum anti Listeria (54). Reagents used were: cycloheximide (Merck), clasto-lactacystin β-lactone (Affinity), Epoxomicin (Affinity), Bafilomycin A1 (Upstate), ubiquitin ladder (poly-ubiquitin chains; Biomol), sulfosuccinimidyl-2-(biotinamido) ethyl-1,3-dipropionate (EZ-link Sulfo-NHS-SS-biotin; Pierce), sodium 2-mercapto sulfonate (MESNA; Fluka), Iodoacetamide (Sigma), immobilized NeutrAvidin (Pierce).

Plasmids. Plasmids codifying for Ha-tagged Cbl, v-Cbl (pMT2SM-Ha-c-Cbl, pMT2Ha-v-Cbl) and pMT2SM-Ha are as in Taher, T. E. I., et al., c-Cbl Is Involved in Met Signaling in B Cells and Mediates Hepatocyte Growth Factor-Induced Receptor Ubiquitination, J. Immunol., vol. 169, pp. 3793-3800 (2002). Plasmids encoding for Ha-Ubiquitin and Ha-monoubiquitin (K29, 48, 63R) are as in Haglund, K., et al., Multiple Monoubiquitination of RTKs is Sufficient for Their Endocytosis and Degradation, Nature Cell Biol., vol. 5, pp. 461-466 (2003).

Assays. Immunoprecipitation, Re-immunoprecipitation, and immunoblots were performed as described before (31). In1B purification was performed as described (54).

Cell surface biotinylation and internalization assay were performed as described before (35). Briefly, the cells were washed in ice-cold PBS, and incubated with 25 mg/ml EZ-link Sulfo-NHS-SS-biotin (Pierce) 15 minutes in PBS pH 8.0, and followed by one wash with serum free media containing 0.1% BSA, followed by one wash in PBS containing Ca and Mg (PBS+CaMg; Gibco). The cells were then warmed to 37° C., incubated with In1B for two minutes, and then cooled down to 4° C. The cells were washed again with PBS+CaMg, and cell-surface debiotinylation was performed by adding 100 mM MESNA in 50 mM Tris-HCl pH 8.6, 100 mM NaCl, 1 mM EDTA and 0.2% BSA (3 times, 10 minutes incubation). The cells were washed with PBS+CaMg, and residual MESNA was inactivated by a ten minute incubation with 120 mM iodoacetamide in PBS. Finally, the cells were then solubilized and precipitated with neutravidin agarose.

Immunoflorescence and differential immunofluorescent labelling of Listeria as well as gentamicin survival assays were performed as described before (54).

RNAi assays used double stranded RNA against: Cbl 5′-GGG AAA AGA MG MU GUA U-3′ (SEQ ID NO:1), cortactin 5′-GGG AGA AUG UCU UUC MG A-3′ (SEQ ID NO:2), Clathrin heavy chain 5′-GGC CCA GGU GGU MU CAU U-3′ (SEQ ID NO:3), Grb2 5′-GGU UUU GM CGA AGA AUG U-3′ (SEQ ID NO:4), eps15 5′-GGU UGA UAC AGG CM UAC U-3′ (SEQ ID NO:5), CIN85 5′-GGA GAG GUU UGU UCC CUG A-3′ (SEQ ID NO:6), GGA3 5′-GGA GGA CUG GGA AUA CAU A-3′ (SEQ ID NO:7), CD2AP 5′-GGA AUG UGA AAA AGC UAC A-3′ (SEQ ID NO:8), AP-50 (AP-2μ chain) 5′-GGA AAA CAU CM GM CM U-3′ (SEQ ID NO:9), and control RNA, (Silencer Negative Control 1 siRNA) were purchased from Ambion. Transfections were performed used oligofectamine (Invitrogen) as recommended by the manufacturer. Cells were tested 72 hours after transfections.

### EXAMPLE 2

### The Listerial Protein In1B Promotes Ubiquitination of its Receptor, Met

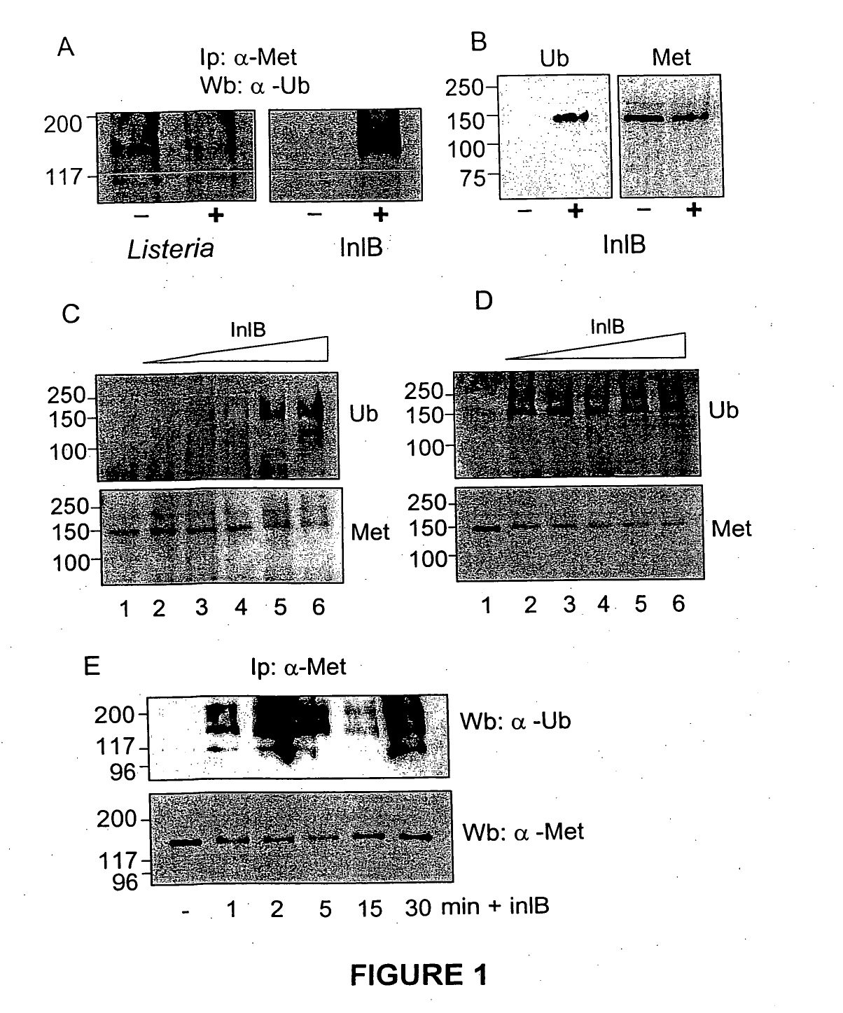

It has been previously described that the ubiquitin ligase Cbl is recruited to the Met complex during the first minute after cell treatment with soluble In1B (31). Whether Listeria infection promotes ubiquitination of Met was studied. Epithelial HeLa cells were infected with Listeria monocytogenes (at a m.o.i. 25) or treated with purified In1B (5 nM) (FIG. 1A) for two minutes. Cellular extracts were immunoprecipitated using an antibody that recognizes Met and ubiquitination of immunoprecipitated proteins was tested with an anti-ubiquitin antibody. A clear increase in the amount of ubiquitinated proteins associated with Met was detected after infection with Listeria. This effect was more dramatic when purified In1B was used (FIG. 1A, right panel), indicating that In1B triggers ubiquitination of Met or Met-associated proteins.

To assess whether the signal observed after In1B treatment corresponded to Met ubiquitination, a re-immunoprecipitation assay was performed. Anti-Met immunoprecipitates from cells samples treated (or not) with In1B were denatured (boiled in presence of 1% SDS), to dissociate Met complexes, and re-immunoprecipitated using anti-Met antibodies. The resulting samples were immunoblotted against ubiquitin (FIG. 1B left panel) and Met (FIG. 1B right panel). Ubiquitin was only detectable in the lane corresponding to In1B treated samples, whereas Met was present in both lanes corresponding to In1B-treated or non-treated samples. Thus, this assay established that In1B induces the ubiquitination of Met. The fact that lower amounts of Met were detected in In1B-treated samples suggested that Met was degraded after its In1B-dependent ubiquitination.

In order to determine the minimal amount of In1B necessary to trigger Met ubiquitination, HeLa cells were treated with increasing amount of In1B for two minutes. Cell extracts were then immunoprecipitated using the anti-Met antibody and blotted with anti ubiquitin or anti Met antibodies (FIGS. 1C and 1D). 1.75 nM of In1B was sufficient to induce ubiquitination of Met. Ubiquitination did not significantly increase using higher amount of In1B (FIG. 1D). Note that ubiquitinated Met migrated slower and than non-ubiquitinated in 8% acrylamide SDS-PAGE gels (FIGS. 1C and 1D), in agreement with an increase in apparent molecular weight of ubiquitinated proteins. Again, Met detection was always weaker after ubiquitination, supporting the previous observation that ubiquitination of Met was leading to Met degradation.

Next, the kinetics of In1B-dependent ubiquitination of Met were tested. In1B (5 nM) was added during increasing time periods to HeLa cells and cell extracts were immunoprecipitated using anti-Met antibodies (FIG. 1E). In1B-induced ubiquitination was maximal two minutes after treatment and declined thereafter. Interestingly, a new wave of ubiquitination was observed 30 minutes after the addition of In1B. In order to determine if the decrease of Met-ubiquitination, observed after five minutes of In1B treatment, was due to degradation of Met, the same samples were analyzed with an anti-Met antibody. In agreement with previous observations, the band corresponding to Met disappeared shortly after In1B treatment, suggesting that the apparently lower level of Met-ubiquitination was due to degradation of Met. Surprisingly, a new band corresponding to Met appeared 15 minutes after the addition of In1B. The appearance of a new wave of Met-ubiquitination suggested that newly synthesized Met could interact with In1B present in the medium and in turn become ubiquitinated.

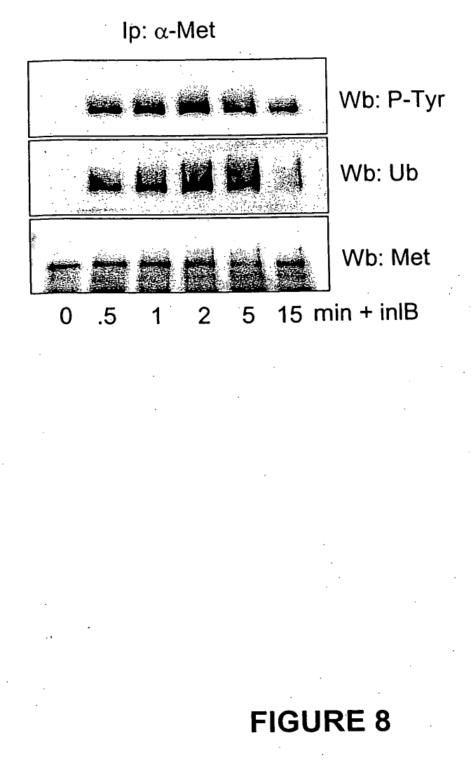

Ligand-induced ubiquitination of RTKs is preceded by phosphorylation of the receptor, which is then recognized and ubiquitinated by the ubiquitin ligase Cbl (33). As previously shown (27), In1B rapidly stimulates Met phosphorylation and the kinetics of ubiquitination of Met follows that of phosphorylation with a maximum at two minutes after stimulation (FIG. 8).

### EXAMPLE 3

### In1B Induces Rapid Degradation and Specific De-Novo Synthesis of Met

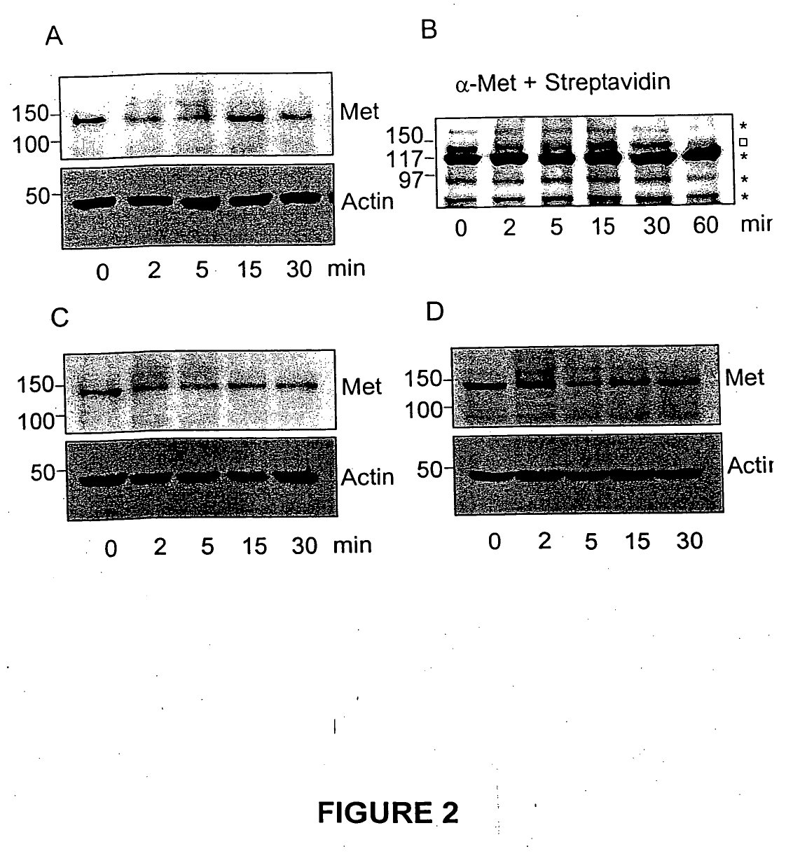

To study In1B-induced degradation of Met in more detail, HeLa cells were treated during increasing periods with In1B (5 nM) and anti-Met immunoblots were performed directly on total cell extracts (FIG. 2A). Two minutes after the addition of In1B, the total amount of Met was strongly reduced. Met degradation was even more acute after five minutes of cell exposure to In1B. After 15 minutes of In1B treatment, the amount of Met was similar to the level of Met in non-treated cells, in agreement with previous observations following immunoprecipitation (FIG. 1E). This variation in protein amount after In1B treatment was specific to Met and did not appear to be a general phenomenon affecting other cellular proteins, as showed by the absence of changes in actin levels (FIG. 2A). This result was confirmed by blotting extracts from HeLa cells treated with 5 nM In1B with anti-Met antibody and Streptavidin, which recognizes cellular proteins unspecifically. As showed in FIG. 2B, the presence of In1B only affected the amount of Met, not the amount of other cellular proteins.

In order to determine whether de-novo synthesis of Met occurred after In1B dependent-degradation, HeLa cells were pre-treated with cycloheximide, which inhibits protein synthesis, for 90 minutes before exposure to In1B. As showed in FIG. 2C, the presence of cycloheximide did not prevent the In1B-dependent degradation of Met, but strongly hampered its de-novo synthesis.

Together these data show that degradation of Met after In1B contact is extremely rapid and also that In1B promotes de-novo and specific re-synthesis of Met. When In1B is present, de-novo synthesized Met was in turn ubiquitinated and then degraded (FIG. 2A). In contrast, if In1B was removed from the medium three minutes after its addition, no degradation of Met was observed after its re-synthesis 15 minutes following the initial exposition of cells to In1B (FIG. 2D).

### EXAMPLE 4

### In1B Promotes Mono-Ubiquitination of Met

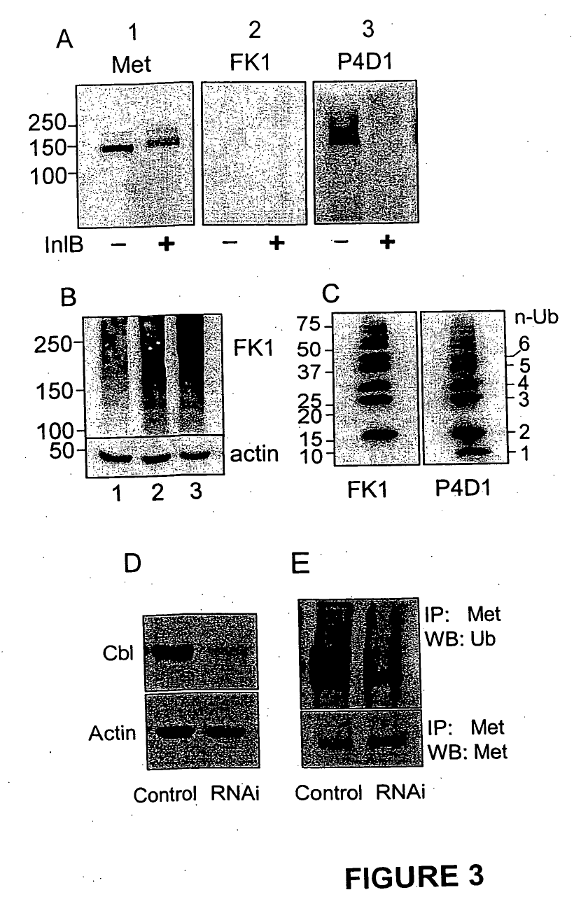

The rapid degradation of Met after In B contact is different from the HGF-dependent degradation, which has been reported to take two hours (19-21), indicating differences in the biochemistry of the process. In addition, HGF-dependent ubiquitination of Met remains unclear, as it has been reported to be both poly-ubiquitination (15,20) or mono-ubiquitination (21). In orderto establish the nature of In1B-dependent ubiquitination of Met, both a monoclonal antibody (mAb), named FK1, which binds poly-ubiquitin but is unable to recognize mono-ubiquitinated proteins (17), and P4D1, a monoclonal antibody that recognizes both mono- and poly-ubiquitinated proteins, were used. Samples from HeLa cells treated with In1B (5 nM) were immunoprecipitated using an anti-Met antibody, and then Met-immunoprecipitates were analyzed with an anti Met mAb (FIG. 3A1), FK1 (FIG. 3A2) or P4D1 (FIG. 3A3). The fact that FK1 did not recognize ubiquitinated Met, whereas P4D1 did, suggests that In1B induces mono-ubiquitination rather than poly-ubiquitination of Met. To test the FK1 functionality, immunoblots of cell samples treated or not with the proteasome inhibitors epoximycin or clasto-lactacystin-β-lactone were performed (FIG. 3B). As expected, stronger signals were detected in lanes corresponding to cells treated with proteasome inhibitors, indicating the proper functionality of FK1. Additionally, an ubiquitin ladder, containing one or more ubiquitin modules, was immunodetected with FK1 or P4D1 mAbs (FIG. 3C). Both monoclonal antibodies recognized polyubiquitin chains, but only P4D1 recognized monoubiquitin. These data strongly established that In1B induces mono-ubiquitination of Met.

### EXAMPLE 5

### In1B Dependent Ubiquitination of Met is Achieved by Cbl

Cbl, the ubiquitin ligase that binds to Met and other RTKs and triggers their ubiquitination and further endocytosis after receptor-ligand contact (5, 15, 34), is recruited to the Met complex shortly after In1B addition to cells (31). These data indicated that the role of Cbl in the In1B-dependent ubiquitination and further degradation of Met should be analyzed further.

Short interfering RNA (siRNA) dramatically diminished Cbl expression, while expression of other cellular proteins, e.g. actin, was not affected (FIG. 3D). Transient Cbl knocked down (Cbl-KO) and control cells were stimulated with In1B. The amount of Met and its ubiquitination, was then examined by immunoprecipitation of Met followed by immunodetection using the corresponding antibodies (FIG. 3F). Both In1B-dependent ubiquitination and degradation of Met were strongly impaired in cells that were transiently knocked down for Cbl, demonstrating that Cbl is responsible for the monoubiquitination and degradation of Met after In1B contact.

### EXAMPLE 6

### In1B Induces Endocytosis and Lysosomal Degradation of Met

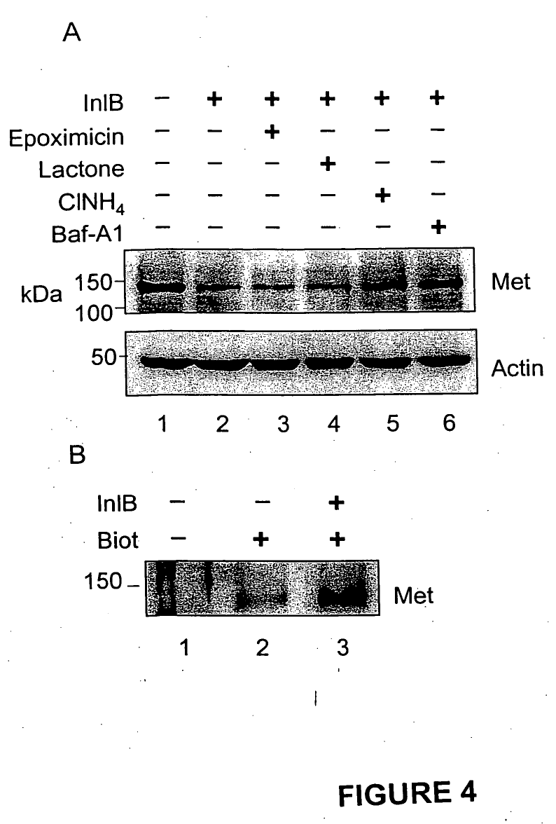

Mono-ubiquitination is the signal that induces endocytosis of RTKs after ligand binding and usually drives cargo proteins to lysosomal degradation (6, 17, 34). To study whether In1B-dependent degradation of Met is a proteasomal or a lysosomal event, HeLa cells were treated with clasto-p-lactacystin lactone or epoximycin, two inhibitors of proteasome degradation, and ammonium chloride or bafilomycin A1, two inhibitors of lysosome degradation, one hour before the addition of In1B. Cellular extracts were then immunoblotted using anti Met antibodies (FIG. 4). The presence of proteasome inhibitors clasto-β-lactacystin lactone or epoximycin did not inhibit In1B-dependent degradation of Met (compare lanes 3 and 4 with lane 2 of FIG. 4). In contrast, the presence of ammonium chloride or bafilomycin A1 (FIG. 4 lanes 5 and 6) significantly protected Met from degradation. These data strongly support the idea that degradation of Met induced by In1B occurs in lysosomes rather that in proteasomes, in agreement with the known lysosomal degradation of mono-ubiquitinated proteins (10).

To study whether In1B does induce endocytosis of Met, a biotin-based assay for internalization of cell surface proteins was used (35). Cell surface proteins were labelled with a biotin derivative that contains a disulphide bond and therefore can be cleaved by reducing agents. After biotin-labelling, cells were treated with In1B and allowed to internalize biotinylated Met. Cells were then treated with MESNA, a reducing agent unable to penetrate into the cell. After cell lysis, biotinylated, i.e. endocytosed proteins, were recovered by precipitation with neutravidin agarose and immunoblotted against Met. As shown in FIG. 4B, the presence of In1B protected Met from de-biotinylation, indicating that In1B induces the endocytosis of Met. These data, together with the results reported above (FIG. 3), definitively establish that In1B induces the mono-ubiquitin dependent endocytosis of Met.

### EXAMPLE 7

### In1B-Induced Degradation of Met During Bacterial Invasion

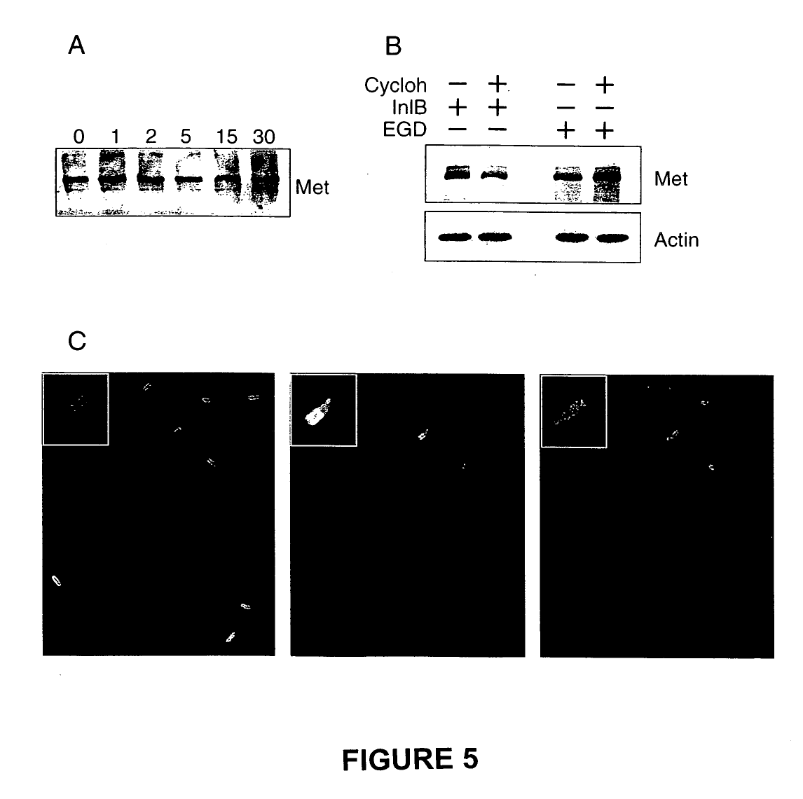

As endocytosis of large particles, i.e. viruses, has been described to be slower than that of macromolecules (36), whether Listeria-induced endocytosis and degradation of Met differs from that following addition of soluble In1B was investigated. First the kinetics of Met degradation after Listeria addition to HeLa cells were followed. As showed in FIG. 5A, Met degradation occurs later, after addition of Listeria (even when a super-invasive strain was used; data not shown), compared to what is observed when soluble In1B is added to the cell culture (FIG. 2A). The effect on Met degradation in Listeria infection was also apparent after cycloheximide treatment, which had no effect after 15 minutes of infection (FIG. 5B). In agreement with these observations, Met strongly localizes with bacteria at the entry sites after ten minutes of bacterial infection, (FIG. 5C).

Since In1B induces the ubiquitin-dependent endocytosis of Met, the localization of Met with entering bacteria suggests a direct coupling of Met endocytosis and bacterial entry.

### EXAMPLE 8

### Cbl Activity is Necessary for Bacterial Endocytosis

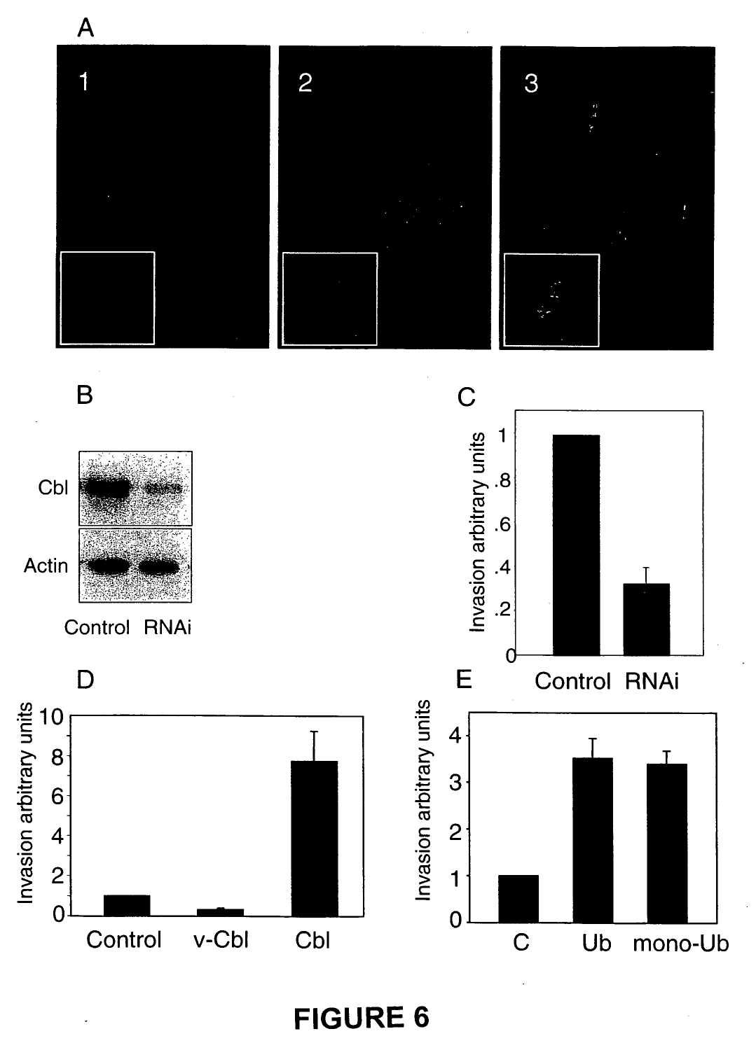

To test this hypothesis, the role of Cbl in bacterial entry was further analyzed. HeLa cells that expressed an Ha-tagged version of Cbl (15) were infected with a superinvasive strain of Listeria (BUG 1641), which overexpress In1B covalently attached to its surface, thus improving the normal signals. The recruitment of Cbl by immunofluorescence was analyzed. These experiments clearly showed (FIG. 6A) that Cbl was recruited to the entry site of Listeria. Next, bacterial entry in cells, in which expression of Cbl was knock down by siRNA was analyzed (FIG. 6B). The effect of Cbl on bacterial infection was monitored using the classical gentamicin survival assay. Transient Cbl-KO or control cells were infected with Listeria for one hour and extracellular bacteria were then killed by adding gentamicin. Intracellular, invasive, bacteria were counted by plating cell lysates. As shown in FIG. 6C, a three-fold decrease in bacterial invasion was detected in Cbl-KO cells, highlighting the critical role of Cbl activity for bacterial infection. The role of Cbl during bacterial invasion was also tested in HeLa cells expressing an Ha-tagged dominant negative form of Cbl (Ha-v-Cbl) (15) lacking the ubiquitin ligase RING domain, using a differential immunofluorescence labelling assay (FIG. 6D). Specifically, the number of intracellular vs. extracellular bacteria was counted after infection. A three-fold decrease in bacterial invasion was detected in perfect agreement with the RNAi experiments. Interestingly, HeLa cells overexpressing wild type Cbl (Ha-tagged Cbl) showed an ˜8 fold increase in Listeria invasion, confirming that Cbl is critical not only for In1B-dependent ubiquitination and endocytosis of Met (FIGS. 3D, 3F), but also for bacterial entry into mammalian cells.

Furthermore, the relevance of ubiquitination in bacterial endocytosis was monitored by differential immunofluorescence labelling assay, on cells overexpressing ubiquitin and a ubiquitin mutant L 9, 48, 63 R only able to form monoubiquitin (17). As showed in FIG. 6E the infection rate was three to four times higher when ubiquitin or monoubiquitin were overexpressed, highlighting the relevance of ubiquitin during Listerial invasion and supporting the observation that Met becomes mono-ubiquitinated.

### EXAMPLE 9

### The Endocytic Machinery is Necessary for Listeria Invasion

The first step in ligand-dependent endocytosis of RTKs after ligand contact is the Cbl dependent ubiquitination of the receptor, followed by recruitment of proteins belonging to the endocytic machinery and formation of a clathrin coated vesicle that is finally endocytosed (6, 10). Besides clathrin and clathrin-adaptors (38), other proteins involved in the ligand-induced endocytosis of Met and other RTKs. These include eps15, a ubiquitin binding protein that may be involved in the recruitment of clathrin to the pits (38), Grb2 an adaptor protein that can direct Cbl to activated receptors and is involved in the cargo entry into the clathrin coated pit (39, 40), dynamin a GTPase that directs the pinching off of the CCV from the membrane (41), CIN85 a Cbl binding protein that, through its interactions with endophilin, can induce membrane curvature (13, 14), CD2AP, a CIN85 related protein that also binds Cbl and cortactin, connecting the Cbl mediated endocytosis to the actin polymerization, as cortactin can activate the arp2/3 complex (42).

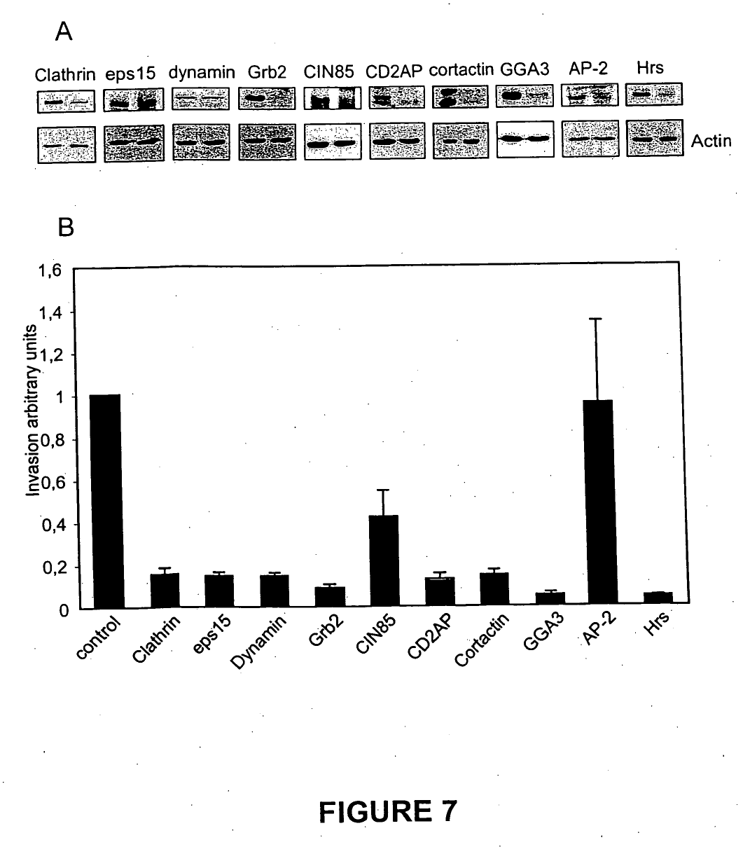

The role of these different components of the endocytic machinery in Listeria entry was examined. RNAi was used to inhibit expression (FIG. 7A), and the effect of the absence of proteins involved in ligand-induced endocytosis of RTKs affected the rate of Listeria invasion was tested. As showed in FIG. 7B, cells with reduced levels of clathrin, eps15, Grb2, dynamin, CIN85, CD2AP, cortactin, as well as cells with reduced levels of the clathrin adaptors GGA3 and Hrs (43) were infected less efficiently by Listeria, demonstrating for the first time the relevance of the clathrin dependent endocytic machinery in Listeria infection. Knock-down expression of the clathrin adaptor complex AP-2 did not affect the endocytosis of Listeria, in agreement with what has been previously shown for the endocytosis of epidermal growth factor receptor (44, 45).

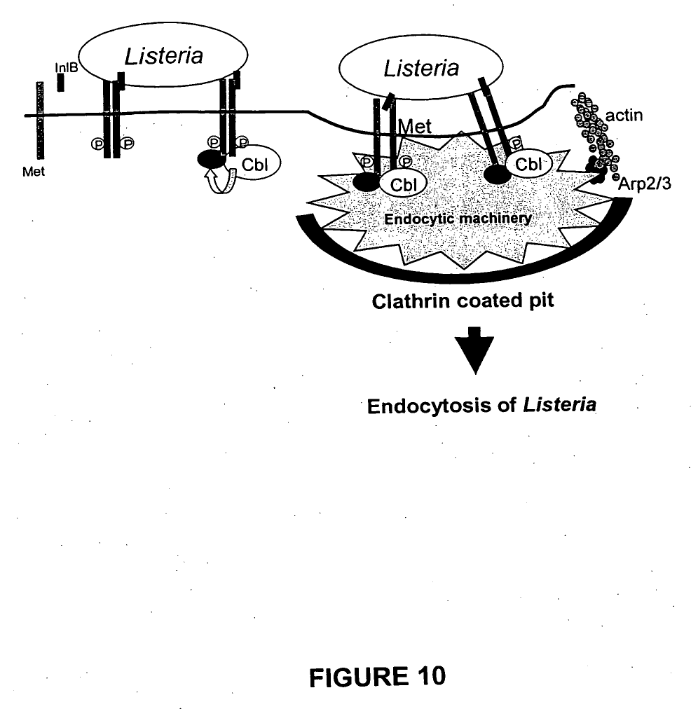

Together, these data revealed that the bacterial protein In1B triggers the Cbl-dependent mono-ubiquitination of Met, coupling Met endocytosis, and Listeria entry into epithelial cells (see FIG. 10).

### EXAMPLE 10

### Maintenance of InIB-Induced Degradation of Met

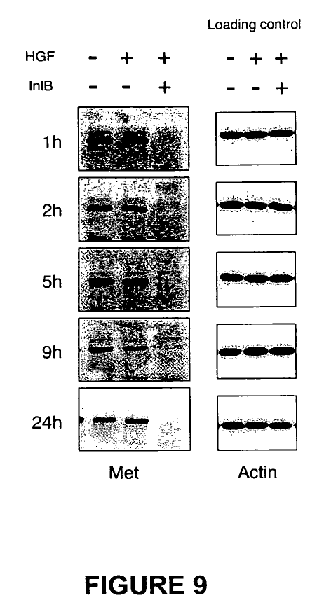

Shortly after InIB treatment, Met is very rapidly degraded. Thus, whether InIB-induced degradation of Met is maintained for longer periods of time was tested. In fact, InIB-induced degradation of Met is maintained in time. FIG. 9 demonstrates that the addition of InIB, even in the presence of HGF, induced Met degradation and that this degradation is maintained from the first hour after the addition of InIB to at least 24 hours.

This result is significant because it shows that InIB degrades Met in the presence of HGF, which mimics the real environment in the body where InIB must act in the presence of HGF. In addition, the result shows that the degradation of Met is maintained during the time, impeding the HGF-Met contact for longer periods of time. Thus, InIB has very specific actions against Met and can be used in anticancer therapies.

### EXAMPLE 11

Whether any differences in the phosphorylation state of specific proteins that are known to be phosphorylated as a consequence of HGF/Met interactions, for example, but not limited to, AKT and ERK, is tested.

In addition, whether InIB is able to inhibit the HGF/Met-dependent proliferation, movement and scattering is tested. Similarly, whether InIB inhibits in vitro the HGF/Met-induced invasion abilities of different cells lines is demonstrated.

In addition, in vivo experiments are performed that include the induction of HGF/Met dependent tumors in mice and the addition of InIB to reduce the size of the tumors and rescue the mice from death. Furthermore, models of HGF/Met dependent metastasis in mice are made and tested to determine if the addition of InIB reduces the number of the metastasis.

### REFERENCES

The entire disclosures of each of the following publications are relied upon and incorporated by reference herein:
1. Trusolino, L. & Comoglio, P. M. Scatter-factor and semaphorin receptors: cell signalling for invasive growth. Nat Rev Cancer 2, 289-300 (2002).
2. Zhang, Y. W. & Vande Woude, G. F. HGF/SF-met signaling in the control of branching morphogenesis and invasion. J Cell Biochem 88, 408-17 (2003).
3. Wang, R., Ferrell, L. D., Faouzi, S., Maher, J. J. & Bishop, J. M. Activation of the Met receptor by cell attachment induces and sustains hepatocellular carcinomas in transgenic mice. J Cell Biol 153, 1023-34 (2001).
4. Bache, K. G., Slagsvold, T. & Stenmark, H. Defective downregulation of receptor tyrosine kinases in cancer. Embo J 23, 2707-12 (2004).
5. Dikic, I., Szymkiewicz, I. & Soubeyran, P. Cbl signaling networks in the regulation of cell function. Cell Mol Life Sci 60, 1805-27 (2003).
6. Marmor, M. D. & Yarden, Y. Role of protein ubiquitylation in regulating endocytosis of receptor tyrosine kinases. Oncogene 23, 2057-70 (2004).
7. Weissman, A. M. Themes and variations on ubiquitylation. Nat Rev Mol Cell Biol 2,169-78. (2001).
8. Schwartz, D. C. & Hochstrasser, M. A superfamily of protein tags: ubiquitin, SUMO and related modifiers. Trends Biochem Sci 28, 321-8 (2003).
9. Kloetzel, P. M. Antigen processing by the proteasome. Nat Rev Mol Cell Biol 2,179-87. (2001).
10. Haglund, K., Di Fiore, P. P. & Dikic, I. Distinct monoubiquitin signals in receptor endocytosis. Trends Biochem Sci 28, 598-603 (2003).
11. Hicke, L. Protein regulation by monoubiquitin. Nat Rev Mol Cell Biol 2, 195-201. (2001).
12. Marchese, A. & Benovic, J. L. Ubiquitination of g-protein-coupled receptors. Methods Mol Biol 259, 299-306 (2004).
13. Petrelli, A. et al. The endophilin-CIN85-Cbl complex mediates ligand-dependent downregulation of c-Met. Nature 416,187-90 (2002).
14. Soubeyran, P., Kowanetz, K., Szymkiewicz, I., Langdon, W. Y. & Dikic, I. Cbl-CIN85-endophilin complex mediates ligand-induced downregulation of EGF receptors. Nature 416, 183-7 (2002).
15. Taher, T. E. et al. c-Cbl is involved in Met signaling in B cells and mediates hepatocyte growth factor-induced receptor ubiquitination. J Immunol 169, 3793-800 (2002).
16. Terrell, J., Shih, S., Dunn, R. & Hicke, L. A function for monoubiquitination in the internalization of a G protein-coupled receptor. Mol Cell 1,193-202 (1998).
17. Haglund, K. et al. Multiple monoubiquitination of RTKs is sufficient for their endocytosis and degradation. Nat Cell Biol 5, 461-6 (2003).
18. Oved, S. & Yarden, Y. Signal transduction: molecular ticket to enter cells. Nature 416,133-6 (2002).
19. Hammond, D. E., Urbe, S., Vande Woude, G. F. & Clague, M. J. Down-regulation of MET, the receptor for hepatocyte growth factor. Oncogene 20, 2761-70 (2001).
20. Kermorgant, S., Zicha, D. & Parker, P. J. Protein kinase C controls microtubule-based traffic but not proteasomal degradation of c-Met. J Biol Chem 278, 28921-9 (2003).
21. Carter, S., Urbe, S. & Clague, M. J. The met receptor degradation pathway: Requirement for K48-linked polyubiquitin independent of proteasome activity. J Biol Chem (2004).
22. Peschard, P., Ishiyama, N., Lin, T., Lipkowitz, S. & Park, M. A conserved DpYR motif in the juxtamembrane domain of the met receptor family forms an atypical c-Cbl/Cbl-b tyrosine kinase binding domain binding site required for suppression of oncogenic activation. J Biol Chem 279, 29565-71 (2004).
23. Finlay, B. B. & Cossart, P. Exploitation of mammalian host cell functions by bacterial pathogens. Science 276, 718-25. (1997).
24. Braun, L., Ohayon, H. & Cossart, P. The InIB protein of Listeria monocytogenes is sufficient to promote entry into mammalian cells. Mol Microbiol 27, 1077-87. (1998).
25. Cossart, P., Pizarro-Cerda, J. & Lecuit, M. Invasion of mammalian cells by Listeria monocytogenes: functional mimicry to subvert cellular functions. Trends Cell Biol 13, 23-31. (2003).
26. Cossart, P. & Lecuit, M. Interactions of Listeria monocytogenes with mammalian cells during entry and actin-based movement: bacterial factors, cellular ligands and signaling. Embo J 17, 3797-806. (1998).
27. Shen, Y., Naujokas, M., Park, M. & Ireton, K. InIB-dependent internalization of Listeria is mediated by the Met receptor tyrosine kinase. Cell 103, 501-10. (2000).
28. Cossart, P. Met, the HGF-SF receptor: another receptor for Listeria monocytogenes. Trends Microbiol 9, 105-7. (2001).
29. Cossart, P. & Bierne, H. The use of host cell machinery in the pathogenesis of Listeria monocytogenes. Curr Opin Immunol 13, 96-103. (2001).
30. Bierne, H. & Cossart, P. In1B, a surface protein of Listeria monocytogenes that behaves as an invasin and a growth factor. J Cell Sci 115, 3357-67. (2002).
31. Ireton, K., Payrastre, B. & Cossart, P. The Listeria monocytogenes protein In1B is an agonist of mammalian phosphoinositide 3-kinase. J Biol Chem 274, 17025-32 (1999).
32. Pizarro-Cerda, J. et al. Distinct protein patterns associated with Listeria monocytogenes InIA- or In1B-phagosomes. Cell Microbiol 4, 101-15 (2002).
33. Dikic, I. & Giordano, S. Negative receptor signalling. Curr Opin Cell Biol 15,128-35 (2003).
34. Hicke, L. & Dunn, R. Regulation of membrane protein transport by ubiquitin and ubiquitin-binding proteins. Annu Rev Cell Dev Biol 19, 141-72 (2003).
35. Hammond, D. E. et al. Endosomal dynamics of Met determine signaling output. Mol Biol Cell 14, 1346-54 (2003).
36. Ehrlich, M. et al. Endocytosis by random initiation and stabilization of clathrin-coated pits. Cell 118, 591-605 (2004).
37. Bierne, H. et al. A role for cofilin and LIM kinase in Listeria-induced phagocytosis. J Cell Biol 155,101-12. (2001).
38. Dupre, S., Urban-Grimal, D. & Haguenauer-Tsapis, R. Ubiquitin and endocytic internalization in yeast and animal cells. Biochimica et Biophysica Acta (BBA)—Molecular Cell Research In Press, Uncorrected Proof (2004).
39. Stang, E. et al. Cbl-dependent Ubiquitination Is Required for Progression of EGF Receptors into Clathrin-coated Pits. Mol Biol Cell 15, 3591-604 (2004).
40. Huang, F. & Sorkin, A. Grb2-mediated Recruitment of the RING Domain of Cbl to the EGF Receptor Is Essential and Sufficient to Support Receptor Endocytosis. Mol Biol Cell (2005).
41. Engqvist-Goldstein, A. E. & Drubin, D. G. Actin assembly and endocytosis: from yeast to mammals. Annu Rev Cell Dev Biol 19, 287-332 (2003).
42. Lynch, D. K. et al. A Cortactin-CD2-associated protein (CD2AP) complex provides a novel link between epidermal growth factor receptor endocytosis and the actin cytoskeleton. J Biol Chem 278, 21805-13 (2003).
43. Owen, D. J., Collins, B. M. & Evans, P. R. Adaptors for clathrin coats: structure and function. Annu Rev Cell Dev Biol 20, 153-91 (2004).
44. Nesterov, A., Carter, R. E., Sorkina, T., Gill, G. N. & Sorkin, A. Inhibition of the receptor-binding function of clathrin adaptor protein AP-2 by dominant-negative mutant mu2 subunit and its effects on endocytosis. Embo J 18, 2489-99 (1999).
45. Motley, A., Bright, N. A., Seaman, M. N. & Robinson, M. S. Clathrin-mediated endocytosis in AP-2-depleted cells. J Cell Biol 162, 909-18 (2003).
46. Puertollano, R. & Bonifacino, J. S. Interactions of GGA3 with the ubiquitin sorting machinery. Nat Cell Biol 6, 244-51 (2004).
47. Raiborg, C. et al. Hrs sorts ubiquitinated proteins into clathrin-coated microdomains of early endosomes. Nat Cell Biol 4, 394-8 (2002).
48. Raiborg, C., Rusten, T. E. & Stenmark, H. Protein sorting into multivesicular endosomes. Curr Opin Cell Biol 15, 446-55 (2003).
49. Conner, S. D. & Schmid, S. L. Regulated portals of entry into the cell. Nature 422, 3744 (2003).
50. Sieczkarski, S. B. & Whittaker, G. R. Dissecting virus entry via endocytosis. J Gen Virol 83, 1535-45 (2002).
51. Meier, O. & Greber, U. F. Adenovirus endocytosis. J Gene Med 6 Suppl 1, S152-63 (2004).
52. de Melker, A. A., van der Horst, G., Calafat, J., Jansen, H. & Borst, J. c-Cbl ubiquitinates the EGF receptor at the plasma membrane and remains receptor associated throughout the endocytic route. J Cell Sci 114, 2167-78 (2001).
53. Cossart, P. & Sansonetti, P. J. Bacterial invasion: the paradigms of enteroinvasive pathogens. Science 304, 242-8 (2004).
54. Pizarro-Cerda, J., Lecuit, M. & Cossart, P. in Methods in Microbiology (eds. Sansonetti, P. J. & A., Z.) 161-177 (Academic Press, 2002).
55. Birchmeier et al., Met, metastisis, mortality and more. Nat. Rev. Mol. Cell Biol., vol. 4, pp. 915-25 (2003).
56. Webb, et al., The geldanamycins are potent inhibitors of the hepatocyte growth factor/scatter factor-met-urokinase plasminogen activator-plasmin proteolytic network. Cancer Res. vol. 60, pp. 341-49 (2000).
57. Banerjee, et al., GW domains of the Listeria monocytogenes invasion protein In1B are required for potentiation of Met activation, Mol. Microbiol., vol. 52, pp. 257-71 (2004)

What is claimed is:

1. A method for treating a carcinogenic tumor in a mammal, comprising:
(A) administering a composition comprising In1B or a fragment of In1B to a patient having a carcinogenic tumor;
(B) achieving degradation of Met in less than one hour; and
(C) reducing at least one carcinogenic aspect of the tumor.

2. The method of claim 1, wherein the carcinogenic aspect of the tumor is tumor growth, proliferation of tumor cells, angiogenesis in the tumor, invasiveness of the tumor, or dedifferentiated tumor morphology.

3. The method of claim 1, wherein the carcinogenic tumor is a tumor of the liver.

4. The method of claim 1, wherein the mammal is a human.

5. The method of claim 1, wherein the fragment of In1B is a peptide from the LRR or GW domain of In1B.

6. An anticancer treatment comprising a pharmaceutical composition comprising In1B or a fragment of In1B.

7. The anticancer treatment of claim 6, wherein the fragment of In1B is a peptide from the LRR or GW domain of In1B.

8. A method of increasing the degradation rate of Met, comprising exposing a cell that contains Met to a composition comprising In1B or a fragment of In1B and increasing the degradation rate of Met in the cell.

9. The method as claimed in claim 8, wherein the fragment of In1B is a peptide from the LRR or GW domain of In1B.

10. A method of screening for compounds that modulate the interaction between Met and In1B, comprising:
(A) adding a compound to a cultured cell;
(B) incubating the cell for different times;
(C) analyzing the Met protein in the cell after the different times of incubation;
(D) determining which compounds alter the rate of degradation of Met in the cell; and
(E) selecting the compounds that alter the rate of degradation of Met as ones that modulate the interaction between In1B and Met.

11. The method of claim 10, wherein the cell is a HeLa cell.

12. A method of screening In1B variants for the ability to interfere with Met signaling comprising:
(A) growing mammalian cells that express Met;
(B) adding medium comprising HGF and at least one In1B variant, or fragment thereof, to a portion of the mammalian cells;
(C) adding medium comprising HGF, but no In1B variant to another portion of the mammalian cells;
(D) detecting and quantifying the amount of phsophorylation of proteins in the portion with only HGF compared to the portion with HGF and the In1B variant; and
(E) identifying the portion that contains In1B variant that reduces the amount of phosphorylation present in the portion without In1B;
wherein, the In1B variants that reduce the amount of phosphorylation of proteins interfere with Met signaling.

13. The method as claimed in claim 12, wherein the phosphorylation of proteins is quantified in the cell using anti-phosphotyrosine antibodies.

14. The method as claimed in claim 12, wherein the phosphorylation of proteins is quantified in specific proteins using antibodies directed to specific phosphoproteins.

15. The method as claimed in claim 14, wherein the specific phosphoprotein is phospho-Met, phospho-ERK, phospho-STAT3, or phospho-46JNK.

16. The method as claimed in claim 12, further comprising a confirmation that the In1B variant interferes with Met signaling by performing an invasion assay, wherein the invasion assay comprises:
(F) growing mammalian cells on a collagen matrix;
(G) adding HGF to induce the cells to separate and invade the collagen matrix;
(H) adding the In1B variant identified in steps (A) through (E) to the culture of invading cells in (G);
(I) inspecting the culture of invading cells in (H); and
(J) determining which In1B variants inhibit the ability of the cells to invade,
wherein the In1B variants that inhibit the ability of the cells to invade interfere with Met signaling.
